(19) United States
(12) Patent Application Publication
(10) Pub. No.: US 2025/0064513 A1
(43) Pub. Date: Feb. 27, 2025

(54) PERICARDIAL TRANSECTION DEVICE WITH SUCTION DEVICE

(71) Applicant: EDWARDS LIFESCIENCES CORPORATION, Irvine, CA (US)

(72) Inventor: Leonardo Paim Nicolau Da Costa, Tampa, FL (US)

(21) Appl. No.: 18/946,456

(22) Filed: Nov. 13, 2024

Related U.S. Application Data
(63) Continuation of application No. PCT/US2023/022972, filed on May 19, 2023.
(60) Provisional application No. 63/346,822, filed on May 27, 2022.

(51) Int. Cl. A61B 18/14; A61B 18/00; A61B 90/00

(52) U.S. Cl. CPC A61B 18/1492; A61B 90/39; A61B 2018/00184; A61B 2018/00273; A61B 2018/00577; A61B 2018/00601; A61B 2018/00625; A61B 2018/1475; A61B 2090/3966

(57) ABSTRACT

A medical device is provided. The medical device includes a multi-lumen catheter. The device also includes an incision device operably coupled to the distal end of the multi-lumen catheter. The incision device includes an incision channel defined along the incision device. The device further includes one or more suction devices adjacent to the incision channel. The device still further includes a cutting surface disposed within the incision channel. The cutting surface is structured to move between a retracted position and a deployed position. In an instance in which the cutting surface is in the retracted position, the cutting surface is disposed within the incision channel. In an instance in which the cutting surface is in the deployed position, the cutting surface at least partially protrudes from the incision channel.

### RELATED APPLICATIONS

This application is a continuation application of International Patent Application No. PCT/US2023/022972, filed May 19, 2023, which claims the benefit of U.S. Provisional Application No. 63/346,822, filed on May 27, 2022. Each of the foregoing patent applications are hereby incorporated by reference in their entireties.

### TECHNICAL FIELD

This disclosure is directed to devices and methods for cutting in the pericardium. The devices and methods are generally applicable to the treatment of heart failure, for example, heart failure with preserved ejection fraction (HFpEF) or reduced injection fraction (HFrEF) by introducing one or more incision lengths in a pericardium, e.g., a parietal layer.

### BACKGROUND

Pericardial restraint is a normal physiologic process that becomes exaggerated, for example, in some patients with heart failure with preserved ejection fraction (HFpEF) and causes the right heart to run out of space when filling, thereby squeezing and over pressurizing the left heart during physical activity in these patients. The increased left heart pressure backs up into the lungs and causes these patients to experience significant breathing difficulties when trying to do minimal activity, (exertional dyspnea). Exertional dyspnea is the most common symptom in patients with HFpEF and the most common cause for admission to the hospital in patients with HF in general. Currently, there is no therapeutic option for patients with HFpEF that specifically targets pericardial restraint.

### SUMMARY

In one example, a medical device for creating elongated incisions within a pericardium is provided. The device includes a multi-lumen catheter including at least one lumen, a longitudinal axis, a proximal end, and a distal end. The device includes an incision device operably coupled to the distal end of the multi-lumen catheter. The incision device includes an incision channel defined along the incision device. The device also includes one or more suction devices adjacent to the incision channel. The one or more suction devices are structured to engage a pericardial tissue. The device further includes a cutting surface disposed within the incision channel. The cutting surface is structured to move between a retracted position and a deployed position. In an instance in which the cutting surface is in the retracted position, the cutting surface is disposed within the incision channel. In an instance in which the cutting surface is in the deployed position, the cutting surface at least partially protrudes from the incision channel.

In some example embodiments, alone or in combination with any of the previous example embodiments, the device also includes a cutting apparatus actuation mechanism structured to move the cutting surface between the retracted position and the deployed position within a human body. In some example embodiments, alone or in combination with any of the previous example embodiments, the one or more suction devices are positioned in a row along the incision channel. In some example embodiments, alone or in combination with any of the previous example embodiments, the one or more suction devices are positioned in a first row and a second row, and each of the first row and the second row are on opposite sides of the incision channel.

In some example embodiments, alone or in combination with any of the previous example embodiments, the first row of the one or more suction device and the second row of the one or more suction devices are parallel. In some example embodiments, alone or in combination with any of the previous example embodiments, the first row of the one or more suction device and the second row of the one or more suction device each have the same number of suction devices. In some example embodiments, alone or in combination with any of the previous example embodiments, the first row of the one or more suction device defines a first row length and the second row of the one or more suction device defines a second row length, and at least one of the first row length or the second row length is the same length as a cutting surface length of the cutting surface.

In some example embodiments, alone or in combination with any of the previous example embodiments, the first row of the one or more suction device defines a first row length and the second row of the one or more suction device defines a second row length, and the first row length and the second row length are the same length as a cutting surface length of the cutting surface. In some example embodiments, alone or in combination with any of the previous example embodiments, the first row of the one or more suction device defines a first row length and the second row of the one or more suction device define a second row length, and at least one of the first row length or the second row length is a greater length than a cutting surface length of the cutting surface.

In some example embodiments, alone or in combination with any of the previous example embodiments, the first row of the one or more suction device defines a first row length and the second row of the one or more suction device define a second row length, and the first row length and the second row length are a greater length than a cutting surface length of the cutting surface. In some example embodiments, alone or in combination with any of the previous example embodiments, the one or more suction devices are structured to receive a suction force upon placement of the catheter within a pericardium of the human body.

In some example embodiments, alone or in combination with any of the previous example embodiments, the suction force is received from a vacuum source connector. In some example embodiments, alone or in combination with any of the previous example embodiments, the vacuum source connector is a tube connected to a vacuum source. In some example embodiments, alone or in combination with any of the previous example embodiments, the vacuum source is a vacuum.

In some example embodiments, alone or in combination with any of the previous example embodiments, the cutting surface is structured to be moveable between the retracted position and the deployed position upon placement of the incision device within a pericardium of the human body. In some example embodiments, alone or in combination with any of the previous example embodiments, the cutting surface is a blade.

In some example embodiments, alone or in combination with any of the previous example embodiments, the cutting surface is an electrode. In some example embodiments, alone or in combination with any of the previous example embodiments, the cutting surface comprises a blade and an electrode. In some example embodiments, alone or in combination with any of the previous example embodiments, the electrode is structured to receive current and/or radio frequency energy to ablate, burn, vaporize, and/or separate tissue.

In some example embodiments, alone or in combination with any of the previous example embodiments, the cutting surface is a wire. In some example embodiments, alone or in combination with any of the previous example embodiments, the cutting surface is a shape-memory wire that is structured to move into the deployed position upon moving a sheath. In some example embodiments, alone or in combination with any of the previous example embodiments, the wire in the deployed position defines a protrusion from the incision channel, the electrode is positioned on the wire adjacent a base of the protrusion.

In some example embodiments, alone or in combination with any of the previous example embodiments, the wire comprises a drawn filled tube. In some example embodiments, alone or in combination with any of the previous example embodiments, the wire includes a core within a shell and the shell is less conductive than the core.

In some example embodiments, alone or in combination with any of the previous example embodiments, the wire includes a core within a shell, and wherein the shell is nitinol and the core is silver. In some example embodiments, alone or in combination with any of the previous example embodiments, the cutting surface comprises a nitinol frame.

In some example embodiments, alone or in combination with any of the previous example embodiments, after an incision is formed within the pericardium, at least a portion of the cutting surface extends through the incision. In some example embodiments, alone or in combination with any of the previous example embodiments, the cutting surface is connected to an energy source connector that electrifies the cutting surface. In some example embodiments, alone or in combination with any of the previous example embodiments, the energy source connector is a wire connected to an energy source. In some example embodiments, alone or in combination with any of the previous example embodiments, the energy source is a battery or generator.

In some example embodiments, alone or in combination with any of the previous example embodiments, the multi-lumen catheter is steerable. In some example embodiments, alone or in combination with any of the previous example embodiments, at least a portion of the multi-lumen catheter is radiopaque. In some example embodiments, alone or in combination with any of the previous example embodiments, the device includes an introducer positioned near the distal end of the multi-lumen catheter. In some example embodiments, alone or in combination with any of the previous example embodiments, at least a portion of the introducer is radiopaque.

In some example embodiments, alone or in combination with any of the previous example embodiments, the at least one lumen comprises a guidewire lumen. In some example embodiments, alone or in combination with any of the previous example embodiments, the device includes a guidewire slidably positioned within the guidewire lumen.

In some example embodiments, alone or in combination with any of the previous example embodiments, the cutting surface is positioned to cut tissue upon being positioned within the human body in an instance in which the cutting surface is in the deployed position. In some example embodiments, alone or in combination with any of the previous example embodiments, the cutting surface when laterally projected, faces away from the distal end and towards the proximal end of the multi-lumen catheter.

In some example embodiments, alone or in combination with any of the previous example embodiments, the cutting surface when laterally projected, is approximately parallel with the multi-lumen catheter. In some example embodiments, alone or in combination with any of the previous example embodiments, the cutting surface when moving between the retracted position and the deployed position, stays approximately parallel with a longitudinal axis along the multi-lumen catheter.

In some example embodiments, alone or in combination with any of the previous example embodiments, the device includes one or more stabilizing members structured to maintain the multi-lumen catheter at a given location within the human body. In some example embodiments, alone or in combination with any of the previous example embodiments, the one or more stabilizing members are structured to move between a stabilizing retracted position and a stabilizing deployed position. In some example embodiments, alone or in combination with any of the previous example embodiments, the one or more stabilizing members are moved from the stabilizing retracted position to the stabilizing deployed position upon positioning of the multi-lumen catheter within the human body.

In some example embodiments, alone or in combination with any of the previous example embodiments, the one or more stabilizing member is a wire, loop, or shape-memory metal. In some example embodiments, alone or in combination with any of the previous example embodiments, the one or more stabilizing member is one or more inflatable structures. In some example embodiments, alone or in combination with any of the previous example embodiments, the one or more stabilizing member are positioned approximately 180 degrees from the cutting surface in an instance in which the cutting surface is in the deployed position.

In some example embodiments, alone or in combination with any of the previous example embodiments, the device includes a sheath structured to moveably cover the incision channel. In some example embodiments, alone or in combination with any of the previous example embodiments, the sheath is structured to cover the incision channel in an instance in which the multi-lumen catheter is not positioned within human body. In some example embodiments, alone or in combination with any of the previous example embodiments, at least a portion of the sheath is radiopaque.

In some example embodiments, alone or in combination with any of the previous example embodiments, at least a portion of a distal end of the sheath is radiopaque. In some example embodiments, alone or in combination with any of the previous example embodiments, the sheath is structured to removably cover the one or more suction devices. In some example embodiments, alone or in combination with any of the previous example embodiments, the sheath is structured to removably cover the one or more stabilizing members.

In some example embodiments, alone or in combination with any of the previous example embodiments, the sheath is structured to removably cover the cutting surface. In some example embodiments, alone or in combination with any of the previous example embodiments, the sheath is structured to removably cover at least two of the one or more suction devices, the one or more stabilizing members, or the cutting surface. In some example embodiments, alone or in combination with any of the previous example embodiments, the sheath is structured to removably cover the one or more suction devices, the one or more stabilizing members, and the cutting surface.

In some example embodiments, alone or in combination with any of the previous example embodiments, sheath includes at least one opening adjacent to a distal end of the sheath. In some example embodiments, alone or in combination with any of the previous example embodiments, the distal end of the sheath is traversable along the multi-lumen catheter to align with the at least one opening of the multi-lumen catheter.

In some example embodiments, alone or in combination with any of the previous example embodiments, at least a portion of the distal end of the sheath and at least a portion of the at least one opening of the multi-lumen catheter are radiopaque to align the distal end of the sheath and the at least one opening of the multi-lumen catheter. In some example embodiments, alone or in combination with any of the previous example embodiments, at least a portion of a circumference of the at least one opening of the sheath and at least a portion of a circumference of the at least one opening of the multi-lumen catheter are radiopaque to align their respective openings.

In some example embodiments, alone or in combination with any of the previous example embodiments, the one or more openings of the sheath are traversable along the multi-lumen catheter allowing an actuator to cause the one or more stabilizing members to laterally project through both the multi-lumen catheter and the sheath. In some example embodiments, alone or in combination with any of the previous example embodiments, the actuator concurrently or sequentially moves the cutting surface and the one or more stabilizing members through the one or more openings of the multi-lumen catheter and the one or more openings of the sheath.

In some example embodiments, alone or in combination with any of the previous example embodiments, at least a portion of cutting surface is reversibly adjustable laterally relative to the longitudinal axis of the multi-lumen catheter between a range of angles. In some example embodiments, alone or in combination with any of the previous example embodiments, a plurality of radiopaque markers is provided along an edge of the incision channel and the position of the cutting surface can be determined based on the radiopaque markers.

In some example embodiments, alone or in combination with any of the previous example embodiments, at least a portion of the cutting apparatus is radiopaque. In some example embodiments, alone or in combination with any of the previous example embodiments, at least a portion of the one or more suction device is radiopaque. In some example embodiments, alone or in combination with any of the previous example embodiments, at least a portion of the one or more stabilizing member is radiopaque.

In some example embodiments, alone or in combination with any of the previous example embodiments, at least a portion of at least two of the cutting apparatus, the one or more suction device, or the one or more stabilizing member are radiopaque. In some example embodiments, alone or in combination with any of the previous example embodiments, at least a portion of the cutting apparatus, the one or more suction device, and the one or more stabilizing member are radiopaque.

In some example embodiments, alone or in combination with any of the previous example embodiments, the cutting surface is structured to have a sufficient force to puncture pericardial tissue in an instance in which the cutting surface is moved from the retracted position to the deployed position. In some example embodiments, alone or in combination with any of the previous example embodiments, the cutting surface is assisted in cutting into the pericardium by the suction force of the one or more suction device. In some example embodiments, alone or in combination with any of the previous example embodiments, the cutting surface is assisted in cutting into the pericardium by a force provided by the one or more stabilizing member.

In some example embodiments, alone or in combination with any of the previous example embodiments, the cutting surface is assisted in cutting into the pericardium by the suction force of the one or more suction device and a force provided by the one or more stabilizing member.

In some example embodiments, alone or in combination with any of the previous example embodiments, at least a portion of the incision device comprises an electrode. In some example embodiments, alone or in combination with any of the previous example embodiments, at least a portion of the cutting surface includes an electrode. In some example embodiments, alone or in combination with any of the previous example embodiments, the electrode is a wire. In some example embodiments, alone or in combination with any of the previous example embodiments, the wire is shaped as one or more arcs projecting laterally through the multi-lumen catheter along the longitudinal axis.

In some example embodiments, alone or in combination with any of the previous example embodiments, the cutting surface includes a scalpel. In some example embodiments, alone or in combination with any of the previous example embodiments, the cutting surface includes a needle. In some example embodiments, alone or in combination with any of the previous example embodiments, the needle of the cutting surface can be moved between the retracted position and the deployed position.

In some example embodiments, alone or in combination with any of the previous example embodiments, at least a portion of the scalpel includes an electrode. In some example embodiments, alone or in combination with any of the previous example embodiments, at least a portion of the needle includes an electrode. In some example embodiments, alone or in combination with any of the previous example embodiments, the at least one electrode is electrically couplable to a source of radiofrequency energy or electrical current sufficient to cut, separate, scissor, or evaporate a portion of the parietal layer.

In some example embodiments, alone or in combination with any of the previous example embodiments, the medical device is sterilized. In some example embodiments, alone or in combination with any of the previous example embodiments, the medical device can be sterilized via gamma sterilization. In some example embodiments, alone or in combination with any of the previous example embodiments, the medical device can be sterilized via ethylene oxide sterilization. In some example embodiments, alone or in combination with any of the previous example embodiments, the medical device can be sterilized via autoclave sterilization. In some example embodiments, alone or in combination with any of the previous example embodiments, the medical device can be sterilized via E-beam sterilization.

In some example embodiments, alone or in combination with any of the previous example embodiments, the incision device includes a rigid housing and a cutting surface received by the rigid housing. In some example embodiments, alone or in combination with any of the previous example embodiments, the device includes at least one nerve detection device. In some example embodiments, alone or in combination with any of the previous example embodiments, the at least one nerve detection device is located on the multi-lumen catheter. In some example embodiments, alone or in combination with any of the previous example embodiments, the at least one nerve detection device is located adjacent the incision device. In some example embodiments, alone or in combination with any of the previous example embodiments, the at least one nerve detection device is located on the puncturing tip. In some example embodiments, alone or in combination with any of the previous example embodiments, the at least one nerve detection device is located on the cutting surface.

In some example embodiments, alone or in combination with any of the previous example embodiments, the device includes at least one nerve stimulation device. In some example embodiments, alone or in combination with any of the previous example embodiments, the at least one nerve stimulation device is located on the multi-lumen catheter. In some example embodiments, alone or in combination with any of the previous example embodiments, the at least one nerve stimulation device is located adjacent the incision device. In some example embodiments, alone or in combination with any of the previous example embodiments, the at least one nerve stimulation device is located on the puncturing tip. In some example embodiments, alone or in combination with any of the previous example embodiments, the at least one nerve stimulation device is located on the cutting surface.

In some example embodiments, alone or in combination with any of the previous example embodiments, a kit includes the medical device, a guidewire, and a puncturing tip is provided.

In another example, a method of creating elongated incisions within a pericardium is provided. The method includes placing a multi-lumen catheter within a human. The multi-lumen catheter includes at least one lumen, a longitudinal axis, a proximal end, and a distal end. The method also includes positioning an incision device within the pericardium. The incision device is operably coupled to the distal end of the multi-lumen catheter and the incision device includes an incision channel defined along the incision device. The method also includes providing a suction force to one or more suction device adjacent to the incision channel. The one or more suction device is structured to engage a pericardium tissue. The method further includes moving a cutting surface disposed within the incision channel from a retracted position to a deployed position. In an instance in which the cutting surface is in the retracted position, the cutting surface is disposed within the incision channel. In an instance in which the cutting surface is in the deployed position, the cutting surface at least partially protrudes from the incision channel. An incision is made into a parietal layer of the pericardium upon movement of the cutting surface into the deployed position.

In some example embodiments, alone or in combination with any of the previous example embodiments, the one or more suction device is engaged with the parietal layer in an instance the incision is made by the cutting surface. In some example embodiments, alone or in combination with any of the previous example embodiments, the creation of the at least one incision length through the pericardium causes a reduction in pressure exerted on a heart by the pericardium. In some example embodiments, alone or in combination with any of the previous example embodiments, the incision length is created in at least one of a parietal layer or fibrous layer of the pericardium. In some example embodiments, alone or in combination with any of the previous example embodiments, the incision length is created in adipose tissue or fat deposits. In some example embodiments, alone or in combination with any of the previous example embodiments, the at least one incision length is along a length or circumference of only the parietal layer of the pericardium. In some example embodiments, alone or in combination with any of the previous example embodiments, the at least one incision length is made in the parietal layer from an anterior to a posterior of a heart. In some example embodiments, alone or in combination with any of the previous example embodiments, the at least one incision length is made in the parietal layer from a posterior base to an apex of a heart.

The In some example embodiments, alone or in combination with any of the previous example embodiments, the at least one incision length is made in the parietal layer from a posterior right atrium to an apex of a heart. In some example embodiments, alone or in combination with any of the previous example embodiments, the at least one incision length is made in the parietal layer from a left ascending aorta to an apex of a heart. In some example embodiments, alone or in combination with any of the previous example embodiments, the at least one incision length is made in the parietal layer from a right ascending aorta to an apex of a heart. In some example embodiments, alone or in combination with any of the previous example embodiments, the at least one incision length is made in the parietal layer transversely about a heart.

In some example embodiments, alone or in combination with any of the previous example embodiments, the method includes, before creating the incision length, puncturing pericardial tissue and providing an access point into a pericardial space. In some example embodiments, alone or in combination with any of the previous example embodiments, the method includes, before puncturing, providing subxiphoid access to the pericardium. In some example embodiments, alone or in combination with any of the previous example embodiments, the method includes, before puncturing, providing transvascular access to the pericardium.

In some example embodiments, alone or in combination with any of the previous example embodiments, the method includes, moving the cutting surface from the deployed position to the retracted position before removing the incision device from the pericardium. In some example embodiments, alone or in combination with any of the previous example embodiments, the method includes, puncturing into the pericardium via an introducer. In some example embodiments, alone or in combination with any of the previous example embodiments, the method includes, after puncturing, inserting a guidewire into the pericardial space. In some example embodiments, alone or in combination with any of the previous example embodiments, the method includes stabilizing the incision device within the pericardium.

In some example embodiments, alone or in combination with any of the previous example embodiments, the incision device is stabilized via one or more stabilization member. In some example embodiments, alone or in combination with any of the previous example embodiments, the one or more stabilizing members are structured to move between a stabilizing retracted position and a stabilizing deployed position. In some example embodiments, alone or in combination with any of the previous example embodiments, the one or more stabilizing members are moved from the stabilizing retracted position to the stabilizing deployed position upon positioning of the multi-lumen catheter within the human body. In some example embodiments, alone or in combination with any of the previous example embodiments, the one or more stabilizing member is a wire, loop, or shape-memory metal.

In some example embodiments, alone or in combination with any of the previous example embodiments, the one or more stabilizing member is one or more inflatable structures.

In some example embodiments, alone or in combination with any of the previous example embodiments, the method includes moving a sheath from a first position covering the incision channel to a second position that uncovers the incision channel. In some example embodiments, alone or in combination with any of the previous example embodiments, the sheath is structured to cover the incision channel in an instance in which the multi-lumen catheter is not positioned within human body. In some example embodiments, alone or in combination with any of the previous example embodiments, the one or more openings of the sheath are traversable along the multi-lumen catheter allowing an actuator to cause the one or more stabilizing members to laterally project through both the multi-lumen catheter and the sheath.

In some example embodiments, alone or in combination with any of the previous example embodiments, the method includes sterilizing the incision device. In some example embodiments, alone or in combination with any of the previous example embodiments, the incision device is sterilized via E-beam sterilization. In some example embodiments, alone or in combination with any of the previous example embodiments, the incision device is sterilized via gamma sterilization. In some example embodiments, alone or in combination with any of the previous example embodiments, the incision device is sterilized via ethylene oxide sterilization. In some example embodiments, alone or in combination with any of the previous example embodiments, the incision device is sterilized via autoclave sterilization.

In some example embodiments, alone or in combination with any of the previous example embodiments, the method also includes determining a location of the incision device within the pericardium via ultrasound. In some example embodiments, alone or in combination with any of the previous example embodiments, creating the at least one incision length is determined in response to a signal indicative of a reduction of restraint of the heart.

In some example embodiments, alone or in combination with any of the previous example embodiments, creating the at least one incision length is determined in response to a signal indicative of a reduction of restraint of the heart; and repeating the creating of the at least one incision length. In some example embodiments, alone or in combination with any of the previous example embodiments, the method also includes, after creating the at least one incision length, confirming a location of a distal end of the multi-lumen catheter device; and in response to a signal indicative of a reduction of restraint of the heart, repeating the steps of creating the at least one incision length, and confirming a location of the distal end.

In some example embodiments, alone or in combination with any of the previous example embodiments, the method also includes moving the cutting surface between the retracted position and the deployed position within a human body. In some example embodiments, alone or in combination with any of the previous example embodiments, each of the one or more suction devices are a suction cup.

In some example embodiments, alone or in combination with any of the previous example embodiments, the vacuum source is provided to each of the one or more suction device. In some example embodiments, alone or in combination with any of the previous example embodiments, the one or more suction device is positioned in a row along the incision channel. In some example embodiments, alone or in combination with any of the previous example embodiments, the one or more suction device is positioned in a first row and a second row, wherein each of the first row and the second row are on opposite sides of the incision channel. In some example embodiments, alone or in combination with any of the previous example embodiments, the first row of the one or more suction device and the second row of the one or more suction devices are parallel.

In some example embodiments, alone or in combination with any of the previous example embodiments, the first row of the one or more suction device and the second row of the one or more suction device each have the same number of suction devices. In some example embodiments, alone or in combination with any of the previous example embodiments, the first row of the one or more suction device defines a first row length and the second row of the one or more suction device defines a second row length, and at least one of the first row length or the second row length is the same length as a cutting surface length of the cutting surface.

In some example embodiments, alone or in combination with any of the previous example embodiments, the first row of the one or more suction device defines a first row length and the second row of the one or more suction device defines a second row length, and the first row length and the second row length are the same length as a cutting surface length of the cutting surface. In some example embodiments, alone or in combination with any of the previous example embodiments, the first row of the one or more suction device defines a first row length and the second row of the one or more suction device define a second row length, and at least one of the first row length or the second row length is a greater length than a cutting surface length of the cutting surface.

In some example embodiments, alone or in combination with any of the previous example embodiments, the first row of the one or more suction device defines a first row length and the second row of the one or more suction device define a second row length, and the first row length and the second row length are a greater length than a cutting surface length of the cutting surface.

In some example embodiments, alone or in combination with any of the previous example embodiments, the suction force is received from a vacuum source connector. In some example embodiments, alone or in combination with any of the previous example embodiments, the vacuum source connector is a tube connected to a vacuum source.

In some example embodiments, alone or in combination with any of the previous example embodiments, the cutting surface is a blade. In some example embodiments, alone or in combination with any of the previous example embodiments, the cutting surface is an electrode. In some example embodiments, alone or in combination with any of the previous example embodiments, the cutting surface comprises a blade and an electrode. In some example embodiments, alone or in combination with any of the previous example embodiments, the electrode is structured to receive current and/or radio frequency energy to ablate, burn, vaporize, and/or separate tissue.

In some example embodiments, alone or in combination with any of the previous example embodiments, the method also includes providing an electrical current to the cutting surface. In some example embodiments, alone or in combination with any of the previous example embodiments, the cutting surface is a wire. In some example embodiments, alone or in combination with any of the previous example embodiments, the cutting surface is a shape-memory wire that is structured to move into the deployed position upon moving a sheath. In some example embodiments, alone or in combination with any of the previous example embodiments, when the wire in the deployed position defines a protrusion from the incision channel, the electrode is positioned on the wire adjacent a base of the protrusion.

In some example embodiments, alone or in combination with any of the previous example embodiments, after an incision is formed within the pericardium, at least a portion of the cutting surface extends through the incision. In some example embodiments, alone or in combination with any of the previous example embodiments, the cutting surface is connected to an energy source connector that electrifies the cutting surface. In some example embodiments, alone or in combination with any of the previous example embodiments, the energy source connector is a wire connected to an energy source. In some example embodiments, alone or in combination with any of the previous example embodiments, the energy source is a battery or generator.

In another example embodiment, a medical device is provided. The medical device includes an incision device operably coupled to a distal end of a catheter. The incision device includes an incision channel defined along the incision device. The medical device also includes one or more suction devices adjacent to the incision channel. The one or more suction devices are structured to engage a pericardial tissue. The medical device further includes a cutting surface disposed within the incision channel. The cutting surface is structured to move between a retracted position and a deployed position. In an instance in which the cutting surface is in the retracted position, the cutting surface is disposed within the incision channel. In an instance in which the cutting surface is in the deployed position, the cutting surface at least partially protrudes from the incision channel.

In some example embodiments, alone or in combination with any of the previous example embodiments, the medical device also includes a cutting apparatus actuation mechanism structured to move the cutting surface between the retracted position and the deployed position.

In some example embodiments, alone or in combination with any of the previous example embodiments, the one or more suction devices are positioned in a row along the incision channel.

In some example embodiments, alone or in combination with any of the previous example embodiments, the one or more suction devices are positioned in a first row and a second row, and each of the first row and the second row are on opposite sides of the incision channel.

In some example embodiments, alone or in combination with any of the previous example embodiments, the first row of the one or more suction device and the second row of the one or more suction devices are parallel.

In some example embodiments, alone or in combination with any of the previous example embodiments, the first row of the one or more suction device defines a first row length and the second row of the one or more suction device defines a second row length, and at least one of the first row length or the second row length is the same length as a cutting surface length of the cutting surface.

In some example embodiments, alone or in combination with any of the previous example embodiments, the first row of the one or more suction device defines a first row length and the second row of the one or more suction device define a second row length, and at least one of the first row length or the second row length is a greater length than a cutting surface length of the cutting surface.

In some example embodiments, alone or in combination with any of the previous example embodiments, the suction force is received from a vacuum source connector.

In some example embodiments, alone or in combination with any of the previous example embodiments, the cutting surface is structured to be moveable between the retracted position and the deployed position upon placement of the incision device.

In some example embodiments, alone or in combination with any of the previous example embodiments, the cutting surface is at least one of a blade or an electrode.

In some example embodiments, alone or in combination with any of the previous example embodiments, the electrode is structured to receive current and/or radio frequency energy to ablate, burn, vaporize, and/or separate tissue.

In some example embodiments, alone or in combination with any of the previous example embodiments, the catheter is steerable.

In some example embodiments, alone or in combination with any of the previous example embodiments, at least a portion of the medical device is radiopaque.

In some example embodiments, alone or in combination with any of the previous example embodiments, the medical device also includes an introducer positioned near a distal end of the incision device.

In some example embodiments, alone or in combination with any of the previous example embodiments, the medical device also includes one or more stabilizing members structured to maintain the incision device at a given location.

In some example embodiments, alone or in combination with any of the previous example embodiments, the medical device also includes a sheath structured to moveably cover the incision channel.

In some example embodiments, alone or in combination with any of the previous example embodiments, the medical device is sterilized.

In some example embodiments, alone or in combination with any of the previous example embodiments, the medical device also includes a controller engaged to the incision device.

In some example embodiments, alone or in combination with any of the previous example embodiments, the controller is structured to provide at least one of suction to the one or more suction devices, movement of the blade between the retracted position and the deployed position, movement of the retractable cutting apparatus along the incision device, energy to the incision device, or movement of one or more stabilizing members.

In another example embodiment, a method of manipulating a medical device is provided. The method includes providing a medical device of any of the embodiments disclosed herein engaged with a controller. The method also includes controlling at least one of supplying suction to the one or more suction devices, movement of the blade between the retracted position and the deployed position, movement of the retractable cutting apparatus along the incision device, supplying energy to the incision device, or movement of one or more stabilizing members.

### BRIEF DESCRIPTION OF THE DRAWINGS

In order to understand and to see how the present disclosure may be carried out in practice, examples will now be described, by way of non-limiting examples only, with reference to the accompanying drawings, in which:

FIG. 1A is a sectional view of a 4-chambered heart.

FIG. 1B is an enlarged view of section 1B of FIG. 1A depicting the layers of the heart wall, including the pericardial cavity.

FIG. 1C is a further enlarged view of section 1B of FIG. 1A depicting the serosal, visceral, fibrous layers and adipose tissue of the parietal pericardium, including the pericardial cavity.

FIG. 2A depicts an example medical device in the retracted position.

FIG. 2B depicts the medical device in the deployed position.

FIG. 3 is an additional view of the medical device.

FIG. 4A illustrates an example medical device positioned within the pericardial cavity as disclosed and described herein.

FIG. 4B illustrates a cutting surface of an example retractable cutting apparatus puncturing a parietal layer of the pericardium.

FIG. 4C illustrates a cutting surface of an example retractable cutting apparatus making the incision along the parietal layer of the pericardium.

FIG. 5 is a simplified diagram of a multi-lumen catheter approach to the pericardial cavity as disclosed and described herein.

FIG. 6 is a simplified diagram of an alternative multi-lumen catheter approach to the pericardial cavity as disclosed and described herein.

FIG. 7 is a simplified diagram of a parietal layer incision length and cut path as disclosed and described herein.

### DETAILED DESCRIPTION

The present disclosure provides for a catheter-based therapy referred to as transcatheter alleviation of pericardial restraint (TAPR) that can reduce pericardial restraint by incising or opening the pericardium with the intention of improving patient outcomes with heart dysfunction, for example, HFpEF or HFrEF and reducing HF readmissions related thereto. The present disclosure, in one example, provides a device with a concealed/medially-facing cutting surface for accessing and modifying a subject's pericardium for relieving pericardial restraint and/or resolving a heart dysfunction. The present disclosure further provides for methods of treating heart dysfunction using the presently disclosed device.

As used herein the phrase “pericardial space” and pericardial cavity are used interchangeably and are inclusive of their ordinary and customary meaning to one of ordinary skill in medical and surgical arts, for example, a space, cavity, or liquid medium generally disposed between the parietal pericardium and visceral pericardium of a mammalian heart.

As used herein the phrase “pericardial tissue” is inclusive of its ordinary and customary meaning to one of ordinary skill in medical and surgical arts, for example, tissue associated with the pericardium.

As used herein, unless otherwise specified, the phrase “parietal layer” comprises at least the serosal and fibrous layer of the parietal pericardium, and optionally adipose tissue contained between, below, above, or within said layers. Further, the phrase “parietal layer” is inclusive of the ordinary and customary meaning to one of ordinary skill in medical and surgical arts, for example tissue layers generally disposed the adjacent to and including adipose tissue within and outside the pericardial cavity and superficial to the visceral layer of the pericardium.

As used herein the phrase “pericardial restraint” includes any terms that signify impaired filling caused by excessive forces from the pericardium.

As used herein the phrase “cutting surface” is inclusive of one or more of an edge of a sharpened blade or the surface of an electrode structured to receive sufficient current or radio frequency energy (RF) to ablate, burn, vaporize, or separate tissue. A cutting surface can be inclusive of both a sharpened edge and an electrode.

As used herein the phrase “reverse cutting” and “pull-back cutting” are used interchangeably and refer to methods involving the presentation of a cutting surface to tissue, the cutting surface adjacent a distal end of a multi-lumen catheter device or catheter, and the application of a directional force sufficient to cut or separate the tissue, the force being substantially in a direction towards the proximal end of the multi-lumen catheter device or catheter, for example, by pulling the multi-lumen catheter device or catheter while the cutting surface is engaged with the tissue.

It should be understood that the term “cutting” used herein refers to tissue disruption, for example, a sharp-cutting incision of the type associated with a knife blade such as a scalpel blade, or an electrosurgical device that provides electrical current to an electrically conductive material or electrode sufficient to disrupt tissue. The term “cutting” used herein includes “filet”, “slicing”, and the like.

As used herein the phrase “incision length” is inclusive of a non-zero distance of a cut or incision, for example, beginning at a first point, e.g., a target point, and terminating at a second point, e.g., an access point. An incision length can be linear, non-linear, or a plurality of linear and/or non-linear lengths that intersect or do not intersect about a curved or non-planar surface, such a heart.

As used herein the phrase “reducing pressure” and “reducing restraint” are inclusive of their ordinary and customary meaning of one to ordinary skill in medical and surgical arts.

As used herein the phrase “reduced ejection fraction” is inclusive of the ordinary and customary meaning to one of ordinary skill in medical and surgical arts, for example, a clinical syndrome in which patients display signs and symptoms of heart failure as the result of high left ventricular (LV) filling pressure despite normal or near normal left ventricle (LV) ejection fraction (LVEF; ≥50 percent).

As used herein the phrase “reduced ejection fraction” is inclusive of the ordinary and customary meaning to one of ordinary skill in medical and surgical arts, for example, impairment of ventricular filling or ejection of blood or both, with a clinical syndrome in which patients display left ventricular ejection fraction (LVEF) of 40% or less and are accompanied by progressive left ventricular dilatation and adverse cardiac remodeling and/or mitral valve dysfunction.

As used herein the phrase “heart dysfunction” is inclusive of the ordinary and customary meaning to one of ordinary skill in medical and surgical arts, for example, heart failure or congestive heart failure.

As used herein the phrase “incision device” is inclusive of a device with a cutting surface, for example an edge of a blade or a surface of an electrode.

As used herein the phrase “pericardial incision assembly” and “incision assembly” are used interchangeable and refer to an assemblage that includes an incision device.

As used herein the phrase “multi-lumen catheter device” is inclusive of a catheter structured with at least one lumen comprising a medical instrument, device, or component thereof, for example, an incision device.

As used herein, the terms “first,” “second,” and the like are only used to describe elements as they relate to one another, and are in no way meant to recite specific orientations of an article or apparatus, to indicate or imply necessary or required orientations of an article or apparatus, to indicate or imply necessary or required configurations of an article or apparatus, or to specify how an article or apparatus described herein will be used, deployed, transitioned from different configurations, or positioned in use.

As used herein, when an element is referred to as being “adjacent” and “coupled” when referring to two structures or layers, the two structures or layers are in proximity with one another with no intervening open space between them.

As used herein, when an element is referred to as being “coupled” or “adjacent” to another element, the two elements or structures are in proximity with one another, however, other elements or intervening elements may be present.

As used herein, when an element is referred to as being “directly coupled” or “directly adjacent” to another element, other elements or intervening elements are not present.

As used herein, term “operably coupled”, includes direct coupling and indirect coupling via another component, element, circuit, or structure and/or indirect coupling between items via an intervening item.

As used herein the phrase “nerve stimulation device” is inclusive of a device capable of applying an electrical potential to a nerve and to cause an observable effect that is directly or indirectly correlated to the applied potential, for example a pacing probe stimulating a phrenic nerve and causing an observable breathing disruption.

As used herein the phrase “nerve detecting device” is inclusive of a device capable of establishing a location or locale of at least part of a nerve and providing location or proximity information with no or substantially no physical effect or stimulus on the nerve, for example, an impedance sensor for detecting an electrical field generated by a nerve and to correlate, directly or indirectly, the location or proximity of the nerve relative to the impedance sensor.

As used herein the term “actuator” is inclusive of a mechanism for triggering an action.

As used herein the term “controller” is inclusive of a device having an actuator.

As used herein the phrase “biasing member” is inclusive of a device configurable in a stored energy state and a released energy state, for example, a spring.

As used herein the phrase “stabilizing member” is inclusive of a device configurable to impart stability and/or securement of a device to or within a structure, such as for example, stabilizing or securing a cutting surface positioned in a pericardial cavity from rolling, twisting, buckling and/or oscillating prior to or during use.

As used herein the phrase “puncturing tip” is inclusive of an atraumatic object suitable for puncturing or penetrating tissue without substantial trauma to or bleeding from the vicinity of the picture or penetration.

Several exemplary transection devices for making incisions through the pericardial membrane or parietal layer of the pericardium are described herein. These examples share the characteristic that they may be delivered intravascularly through the right atrial appendage (RAA), Inferior Vena Cava (IVC), Superior Vena Cava (SVC), Coronary Sinus (CS), Right Atrium (RA), Left Atrial Appendage (LAA), or Right Ventricle (RV). Alternatively, devices may be delivered to the pericardial cavity through catheter access via a subxiphoid approach.

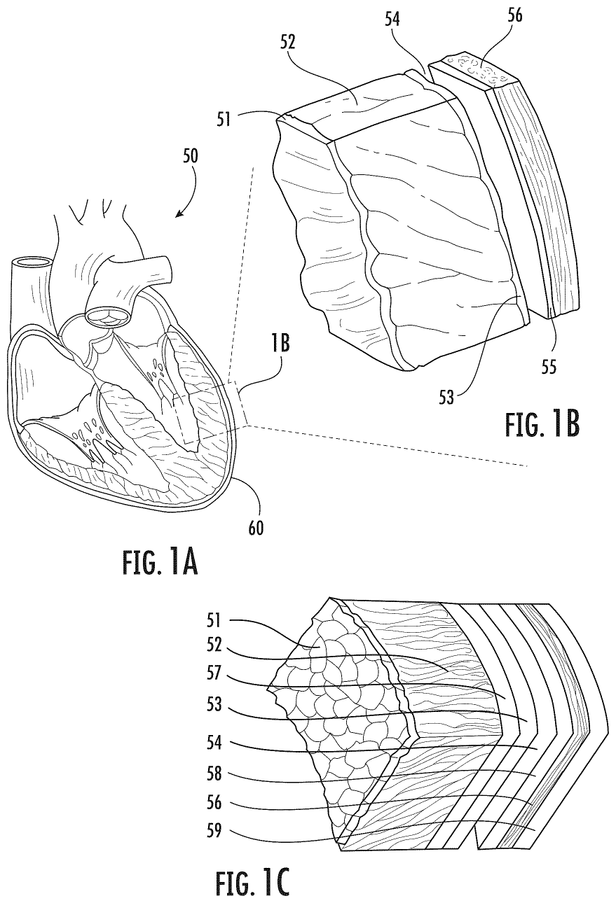

With reference to FIGS. 1A, 1B, 1C, and section 1B, layers of a heart wall of a heart 50, from inside-out, being the endocardium 51, the myocardium 52, epicardial adipose tissue 57, the visceral layer 53 of the serous pericardium, the pericardial cavity 54, the parietal layer 55 of the serous pericardium 58, and the fibrous pericardium 56, and pericardial adipose tissue 59 are depicted. In one example, the presently disclosed devices are structured for introduction to the pericardial cavity 54 and for cutting tissue layers generally disposed adjacent to and including adipose tissue within and outside the pericardial cavity and superficial to the visceral layer 53 of the pericardium.

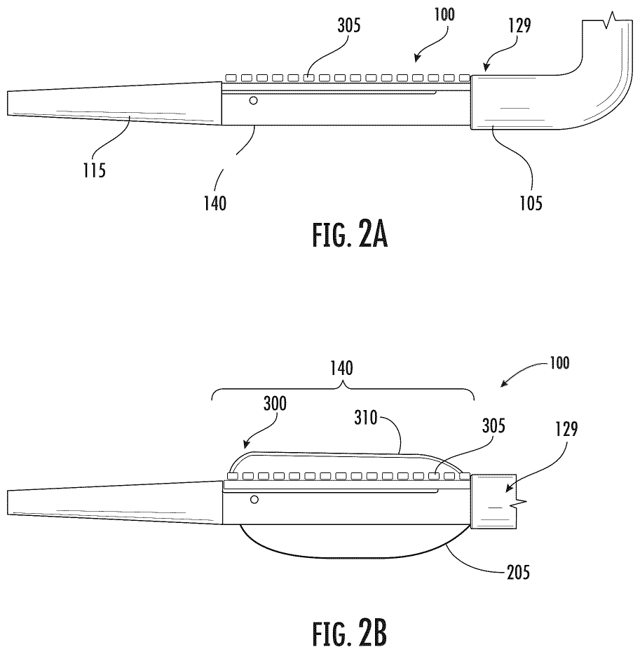

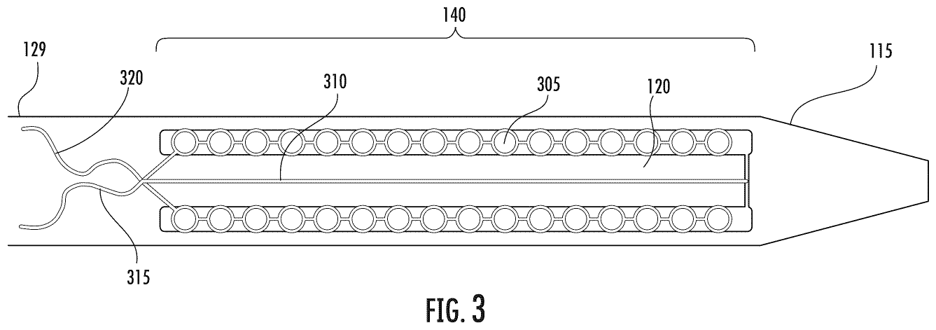

Referring now to FIGS. 2A, 2B, and 3, in one example the presently disclosed retractable cutting apparatus 300 comprises a flexible catheter 129 comprising a distal end, at least one lumen, and a longitudinal axis; an incision device 140 coupled to the distal end of the catheter; and an introducer 115 coupled to and projecting from the incision device 140 In one example, the introducer 115 comprises a puncturing tip. In one example, at least a portion of the flexible catheter 129 tip is radiopaque. In one example, at least a portion of the incision assembly tip (e.g., the sharp edge of cutting surface 310) is radiopaque. In one example, at least a portion of the introducer 115 tip is radiopaque.

The incision device 140 defines an incision channel 120 (shown in FIG. 3) that is structured to receive a retractable cutting apparatus. While the cutting surface 310 of the retractable cutting apparatus is shown in FIG. 2B, various other retractable cutting apparatuses discussed herein can also be disposed within the incision channel. The retractable cutting apparatus can be moved along the incision channel via an internal actuator (e.g., cutting apparatus movement actuator). Additionally, the cutting surface 310 of the retractable cutting apparatus 300 may be held in the retracted position via a sheath (e.g., the retractable cutting apparatus 300 may be formed out of a memory-shape material that protrudes out of the incision channel in an instance in which the sheath is not covering the retractable cutting apparatus).

FIG. 2A illustrates the cutting surface 310 of the retractable cutting apparatus 300 in the retracted position. As discussed herein, the cutting surface 310 is structured to move between a retracted position and a deployed position. In the retracted position, the cutting surface 310 is disposed within the incision channel (e.g., not protruding from the incision channel). In the deployed position, the cutting surface 310 at least partially protrudes from the incision channel. As such, in the deployed position, the cutting surface 310 is exposed from the catheter.

FIG. 2B illustrates the cutting surface 310 of the retractable cutting apparatus 300 in the deployed position. As discussed herein, the cutting surface 310 is structured to move between a retracted position and a deployed position. In the retracted position, the cutting surface 310 is disposed within the incision channel (e.g., not protruding from the incision channel). In the deployed position, the cutting surface at least partially protrudes from the incision channel. As discussed herein in more detail, the device 100 may have one or more stabilizing members 205 that are structured to maintain the position of the incision device 140 when deployed.

Various embodiments of the medical devices discussed herein may have stabilizing members 205. The stabilizing member 205 may be independently user controlled by advancing actuating wire distally toward introducer 115 tip that laterally extends member 205 a distance from the multi-lumen catheter. In one example, two or more stabilizing members 205 are positioned radially about the assembly. In one example, two or more stabilizing members 205 are positioned radially about the assembly about 120 degrees apart. In one example, the two or more stabilizing members 205 are offset longitudinally from the cutting surface to minimize or eliminate pushing the device through the newly cut slit in the parietal layer 55 of the pericardium 60 just as it is formed. Stabilizing members 205 can be flexible rods or strip, or inflatable structures, such as balloons that can be inflated with air or liquid (saline).

The one or more stabilizing members 205 are structured to move between a stabilizing retracted position and a stabilizing deployed position upon positioning of the multi-lumen catheter within the human body.

The medical device 100 may also include a sheath structured to moveably cover the incision channel. The sheath is structured to cover the incision channel in an instance in which the multi-lumen catheter is not positioned within human body. Once the multi-lumen catheter has been positioned (e.g., the incision device is positioned where the cut in the parietal layer 55 of the pericardium 60 is to be made), the sheath may be actuated to uncover the incision channel. In some embodiments, the sheath may not be moveable and merely has a slit to allow the knife to extrude. The sheath can also hold the one or more stabilizing members in the stabilizing retracted position during movement (e.g., the sheath moves to uncover the stabilizing member(s) upon placement in the pericardium.

As shown in FIG. 3, the incision device 140 includes a retractable cutting apparatus 300. The retractable cutting apparatus 300 includes one or more suction devices 305 and a cutting surface 310. The one or more suction devices 305 may be a suction cup that is structured to engage with the wall of the pericardial cavity 54. As shown, the one or more suction devices 305 may include one or more rows of suction devices adjacent to the incision channel 120. For example, the medical device may have two rows on opposite sides of the incision channel 120. Each of the rows of suction devices 305 may be at least the length of the cutting surface 310 (e.g., the suction devices 305 may line the incision channel for at least the length of the cutting surface 310).

The rows of the suction device(s) 305 may be folded on one another for transport. For example, the suction device(s) 305 may be folded in an instance in which the sheath is covering the suction device(s) 305.

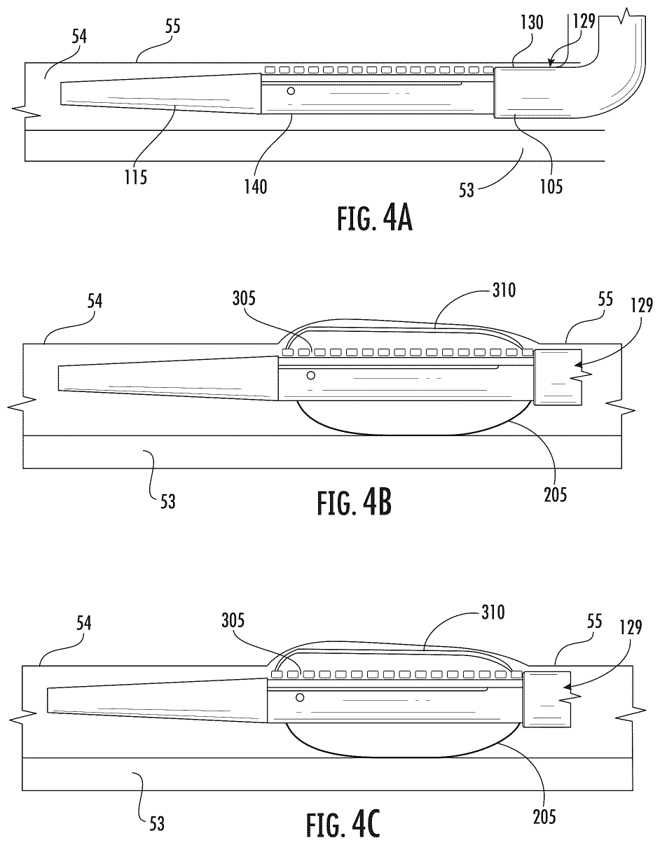

The one or more suction devices 305 may be connected to a vacuum source via a vacuum source connector 320. The vacuum source connector 320 provides the one or more suction devices 305 with a suction force that engages the wall of the pericardial cavity. The suction force may be sufficient to maintain the position of the catheter 129 during the cutting process (as discussed in reference to FIGS. 4A-C). The stabilizing members 205 shown in FIG. 2B may also assist the one or more suction devices 305 to maintain connection with the pericardial cavity (e.g., the stabilizing member 205 may engage the opposite wall of the pericardial cavity).

Materials suitable for the suction devices 305 can include, without limitation, thermoplastic elastomers, EPDM rubbers, thermoset rubbers, polysilicones, polysiloxanes, polyurethanes, polyvinyl chlorides, styrene-ethylene-butylene-styrenes, and polytetrafluoroethylenes, derivatives, copolymers, and blends thereof. For example, the suction device(s) 305 may be a suction cup(s) made out of silicone.

The cutting surface 310 may be any means of causing an incision into the parietal layer 55. The cutting surface 310 may be a needle that is moved between the retracted position and the deployed position. The needle, in such an embodiment, may include a sharp edge that is structured to puncture the parietal layer 55 of the pericardium. The cutting surface 310 may also be a blade that is structured to cause the incision. The needle may be moved through a needle lumen of the catheter 129. The needle may be curved to engage the parietal layer 55. The needle may be advanced along the needle lumen upon placement of the incision device within the pericardial cavity 54. The catheter 129 may also have a wire lumen with a guidewire that provides additional stability to the catheter and the incision device.

The cutting surface 310 may be straight in order to cause a linear incision. The suction device(s) 305 maintain the position of the incision device 140 to allow the incision to be linear. In some instances, the sharp edge of the cutting surface 310 may be curved to allow the blade to puncture pericardium tissue (e.g., puncture into the parietal layer 55) when being moved into the deployed position (e.g., the cutting surface 310 is curved in the direction of the parietal layer 55). The cutting surface 310 is structured to have a sufficient force to puncture pericardial tissue in an instance in which the cutting surface 310 is moved from the retracted position to the deployed position. The cutting surface 310 when laterally projected (e.g., in the deployed position), may face away from the distal end and towards the proximal end of the multi-lumen catheter (e.g., in an instance in which the cutting surface 310 is curved).

Additionally or alternatively, the cutting surface 310 may be an electrode structured to receive sufficient current or radio frequency energy (RF) to ablate, burn, vaporize, or separate tissue. For example, as shown in FIG. 3, the cutting surface 310 may be connected to an electrical source via an electrical source connector 315. The electrical current provided by the cutting surface 310 may be turned off and on via a controller (e.g., the electrical current provided to the cutting surface 310 may be provided upon positioning of the catheter within the pericardial cavity 54). In some embodiments, the cutting surface 310 may have both a sharpened edge and an electrode.

The cutting surface 310 is fixed along the longitudinal axis, such that the cutting surface 310 only moves along the latitudinal axis. The cutting surface 310 may be moved between the retracted position and deployed position manually (e.g., the cutting surface 310 may have an actuator attached to the cutting surface 310 that moves the cutting surface 310 along the latitudinal direction (e.g., to be exposed from the catheter in the deployed position). The cutting surface 310 may also be restricted in moving by a sheath, that maintains the position of the cutting surface 310 within the incision channel. Upon moving the sheath (e.g., along the catheter 129 in the direction of the proximal end of the catheter 129), the cutting surface 310 is no longer restricted and expands into the deployed position. In such an embodiment, the cutting surface 310 may be a shape-memory material and/or spring actuated.

In various embodiments, a controller may be provided to actuate the various mechanisms discussed herein. For example, the controller may be a handle with one or more actuating knobs and/or buttons for controlling the catheter and/or components therein. For example, the controller may actuate the vacuum source provided to the one or more suction devices 305, actuate supply of the electrical current provided to a cutting surface 310 (e.g., via the electrical source connector 315), actuate movement of the retractable cutting apparatus 300, actuate movement of the catheter, actuate movement of the one or more stabilizing members and/or the like.

A method of manipulating medical device 100 would include providing medical device 100 engaged with a controller and controlling, for example, manipulating any one or combination of actuating knobs and/or actuating buttons to provide at least one of suction to the one or more suction devices, movement of the blade between the retracted position and the deployed position, movement of the retractable cutting apparatus along the incision device, supply of energy to the incision device, or movement of one or more stabilizing members with the controller. The aforementioned method is applicable to any previously disclosed transection devices.

One or more components of the medical device may be radiopaque to allow the medical device to be seen within the human body using ultrasound. In various embodiments, portions of the retractable cutting apparatus, multi-lumen catheter, and/or introducer may be radiopaque.

Part or all of the medical device may be sterilized for use. The medical device may be sterilized using various sterilizing techniques, such as E-Beam sterilization, gamma sterilization, ethylene oxide sterilization, autoclave sterilization, aseptic manufacturing/packaging and the like. Additionally, one or more materials used in the medical device may have anti-bacterial characteristics or an antibacterial coating.

The pericardial transection device and/or catheter and/or sheath can be structured such that the total outer diameter (O.D.) introduced to the pericardial cavity is between about 6 Fr (2 mm) and about 30 Fr (10 mm). The pericardial transection device and/or catheter and/or sheath can be structured such that the total outer diameter (O.D.) introduced to the pericardial cavity is between about 6 Fr (2 mm) and about 20 Fr (6.67 mm). The pericardial transection device and/or catheter and/or sheath can be structured such that the total outer diameter (O.D.) introduced to the pericardial cavity is between about 6 Fr (2 mm) and about 15 Fr (5 mm). The pericardial transection device and/or catheter and/or sheath can be structured such that the total outer diameter (O.D.) introduced to the pericardial cavity is between about 6 Fr (2 mm) and about 12 Fr (4 mm). The pericardial transection device and/or catheter and/or sheath can be structured such that the total outer diameter (O.D.) introduced to the pericardial cavity is approximately 10 Fr (3.33 mm).

FIGS. 4A-4C illustrate medical device 100 being deployed. In one example, device 100 is deployed subxiphoidally, e.g., through percutaneous access achieved by directing a needle into the patient under the xiphoid process towards the pericardial cavity or via a surgical incision of the chest. In another example, device 100 is deployed intravascularly thru the right ventricle. The medical device 100 can be advanced through a subject's pericardial cavity 54 in an undeployed configuration with the cutting surface 310 in the retracted position within the incision channel.

The initial access to the pericardial cavity 54 may be achieved via the introducer 115. As the medical device is positioned within the pericardial cavity 54, the sheath 130 may be covering the incision channel. As the multi-lumen catheter is positioned within the pericardial cavity 54 at the location of the incision, the sheath 105 may be removed, as shown in FIG. 4A. The sheath may uncover the incision device 140 that is still in the retracted position.

As shown in FIG. 4A, the one or more suction devices 305 are structured to engage with the wall of the pericardial cavity 54 (e.g., the parietal layer 55). Upon removing the sheath (in an instance in which a sheath is provided), the one or more suction devices 305 receive the suction force from the suction source, as discussed above in reference to FIG. 3. The one or more suction devices 305 are structured to engage the pericardial tissue and maintain the incision device 140 relative to the wall of the pericardial cavity 54 (e.g., as shown in FIG. 4A, the incision device 140 may be approximately parallel with the parietal layer 55).

As shown in FIG. 4B, the one or more stabilizing members 205 may also be deployed engage the visceral layer 53 (e.g., on the opposite wall of the pericardial cavity from the suction device(s) 305). The stabilizing member(s) 205 also maintain the catheter position within the pericardial cavity 54. The stabilizing member 205 may be independently user controlled by advancing an actuating wire distally toward introducer 115 tip that laterally extends stabilizing member(s) 205 a distance from medical device 100. Stabilizing members 205 can be flexible rods or strip, or inflatable structures, such as balloons that can be inflated with air or liquid (saline).

Upon positioning of the incision device 140 within the pericardial cavity 54, the cutting surface 310 may be moved from the retracted position to the deployed position as discussed herein. The cutting surface 310, using a blade, an electrode, and/or the like, punctures into the pericardial tissue allowing the incision to be made along the pericardium tissue (e.g., the parietal layer 55). The incision length may be based on the length of the cutting surface 310. As shown, the catheter 129 maintains the same position within the pericardial cavity 54 due to the suction device(s) and/or the stabilizing member(s) 205. As such, the incision may be a linear incision that is the length of the cutting surface 310 that is engaging the parietal layer 55. The multi-lumen catheter 129 remains stationary within the pericardial cavity 54 during the incision by the cutting surface.

At least a portion of the cutting surface 310 can serve as a radiopaque marker indicating ‘bunching up’ of tissue, for example, due to excessive pull before a through-cut is achieved. In one example, the back silhouette of the cutting surface may be selectively insulated (as it may be activated to achieve the exit puncture) to avoid ablation damage to neighboring anatomy.

Upon completing the intended incision, the cutting surface 310 is moved back to the retracted position. The stabilizing member(s) 205 may be moved to the stabilizing retracted position. The suction force provided to the suction device(s) 305 may be reduced to cause the suction between the suction device(s) and the parietal layer to dissipate. The sheath can be moved to cover the incision channel. The multi-lumen catheter can then be removed from the human body without having any blades exposed.

In one example, a puncture to deliver a guidewire into the pericardial space is performed through heart tissue. When a transvascular approach through the RAA, IVC, or SVC is employed, a closure device may be subsequently introduced for hemostasis at the conclusion of the procedure. In one example, the closure device includes outward or radially directed splines deployed in an expanded configuration. When the guide catheter is removed, the splines or radial members of the closure device contract inwardly towards the unstressed state of the transection device in order to close, occlude, and/or seal the opening. The closure device is designed such that a pericardial cutting device can pass through and into the pericardial space.

In one example, to provide orientational stability of the cutting surface to that of the parietal layer, an OTW introduction is employed for any of the previously disclosed devices, for example, whether through a dedicated lumen in multi-lumen catheter cross-section or ‘Rapid Exchange’ style catheter, or off-center attached cannula, or deflect-resistant catheter. In one example, the delivering catheter comprises radiopaque material randomly distributed or arranged in a pattern for visualization using conventional visualization techniques during use.

Current ECHO/fluoroscopy may not provide the required visualization for certain access applications of the presently disclosed transection devices, for example, gaining guidewire access to the pericardial cavity consistently and repeatedly may be desired. Thus, in one example, the multi-lumen catheter 129 coupled to the presently disclosed transection devices comprises direct visualization allowing the user to watch real-time the advancement of any of the presently disclosed transection devices traverse through various tissue layers until the desired location is reached. Changes in tissue layers that may not be visible under ECHO/fluoroscopy may be easily distinguishable under direct visualization such as tissue/bloodstream (vessel access), myocardium/pericardium (pericardial cavity access), myocardium/pericardium (outside pericardium), among other anatomical features.

Thus, in one example, the presently disclosed devices discussed above further comprise an optical channel in the multi-lumen catheter to accommodate a lens coupled to a fiber optic cable, optionally with a light source, e.g., an LED. In one example, the presently disclosed method further comprises obtaining visual information during accessing, traversal of the pericardial cavity, exiting and/or cutting, for example, using an optical channel in the multi-lumen catheter to accommodate a lens coupled to a fiber optic cable, optionally with a light source, e.g., an LED.

In one example, a puncture to deliver a guidewire into the pericardial cavity 54 is performed through heart tissue in a transvascular approach. When a transvascular approach through the RAA, IVC, or SVC is employed, a closure device (e.g., occluder) may be subsequently introduced for hemostasis at the conclusion of the procedure. In one example, the closure device includes outward or radially directed splines deployed in an expanded configuration. When the guide catheter is removed, the splines or radial members of the closure device contract inwardly towards the unstressed state of the transection device in order to close, occlude, and/or seal the opening. The closure device is designed such that a pericardial cutting device can pass through and into the pericardial space.

The following exemplary occlusion descriptions relate to a transvascular approach through the RAA, IVC, SVC, or CS using one of the aforementioned transection devices (e.g., incision device 140). In one example, an introducer/dilator 115 delivers a wire into the pericardial space through heart tissue. A closure or occlusion device is introduced for hemostasis during the procedure. The closure or occlusion device in one example includes outward or radially directed splines deployed in an expanded configuration. When the guide catheter is removed, the splines or radial members of the closure device contract inwardly towards the unstressed state of the transection device in order to close and seal the opening. The closure device is designed such that a pericardial cutting device can pass through and into the pericardial space.

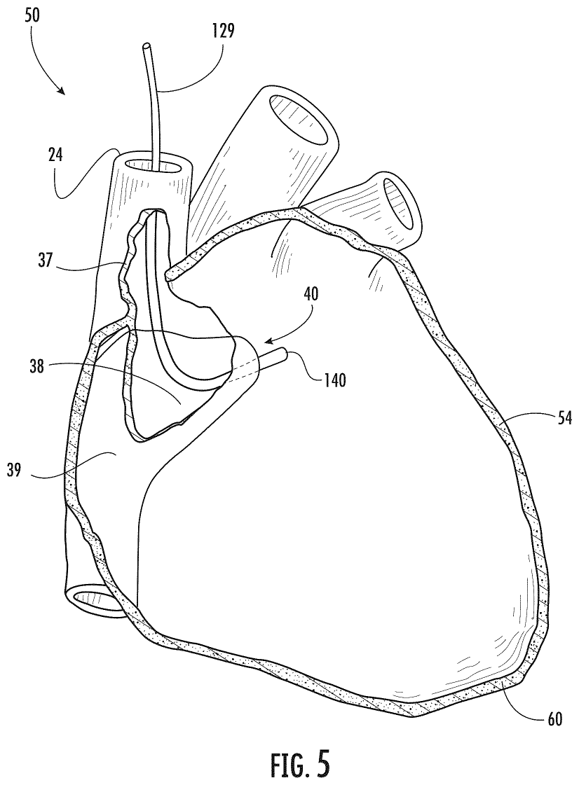

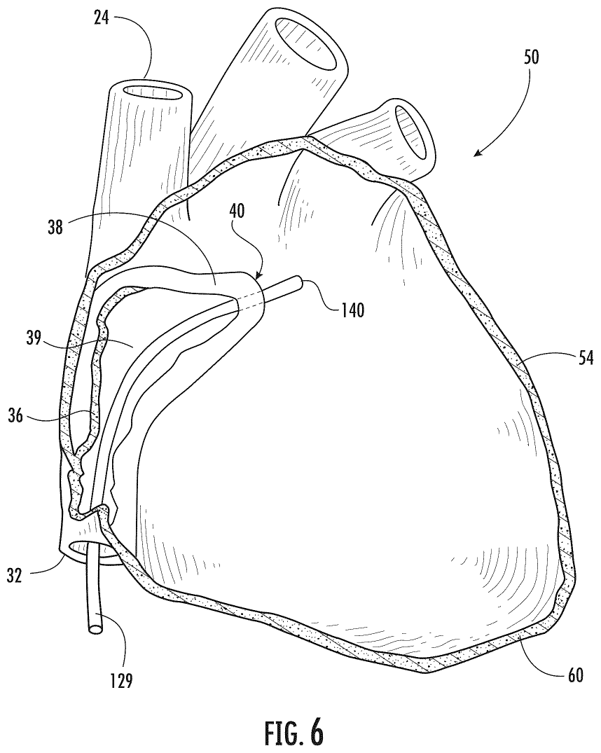

FIGS. 5 and 6 shows exemplary intravascular approaches for delivering the transection devices of the present disclosure to the pericardial cavity 54. Thus, FIG. 5 depicts heart 50 viewed in isolation from the body, with the pericardium 60 or pericardial sac encasing the cardiac muscle (i.e., epicardium, myocardium and endocardium). The small space which is present between the heart muscle and pericardium 60 represents the pericardial cavity 54.

The presently disclosed transection devices that can be presented to the pericardial cavity 54. In one example via the right atrial appendage 38 (RAA), which is a suitable site for entry into the pericardial cavity 54, is used. Right atrial appendage 38 lies tangential to and between pericardium 60 and the epicardium/epicardial adipose tissue 57. In one example, any of the presently disclosed devices is guided into right atrial appendage 38 via right atrium 39 so as to be positioned substantially in parallel with the wall of pericardium 60 such that when the wall of right atrial appendage 38 is pierced by any of the presently disclosed devices substantially without risk of damaging the epicardium or other heart tissue. Other access routes to the pericardial cavity can be used, for example, direct “puncture out” of SVC or IVC/coronary sinus (CS) and a “puncture into” the pericardium.

In some examples, right atrial appendage 38 is accessed via conventional vena cava routes. FIG. 5 illustrates entry of any of the presently disclosed devices into right atrium 39 via the superior vena cava 24 (SVC). A cut-away 37 shows passage of any of the presently disclosed devices through superior vena cava 24, right atrium 39, and right atrial appendage 38. A distal tip of catheter 129 is shown exiting right atrium 39 at apex 40.

FIG. 6 illustrates an alternative entry of any of the previously disclosed devices into right atrium 39 via the inferior vena cava 32 (IVC). A cut-away 36 shows passage of catheter 129 through inferior vena cava 32, right atrium 39, and right atrial appendage 38. A distal tip of catheter 129 is shown exiting right atrium 39 at apex 40.

Thus, by way of example, a method of reducing pericardial restraint of a subject in need thereof using any of the presently disclosed devices is provided by the following steps. Any of the presently disclosed devices is maneuvered through one of the vena cava 24, 32 to right atrium 39. Once inside right atrium 39, any of the presently disclosed devices is passed into the right atrial appendage 38. The wall of right atrial appendage 38 is pierced at apex 40, and the catheter is advanced into the pericardial cavity 54. Other transvascular-right heart routes to the pericardial cavity 54 are envisaged. Furthermore, left atrial appendage, coronary sinus, and right ventricle pathways are envisaged for transvascular access to the pericardial cavity 54.

Note that the wall of the right atrial appendage may be pierced with any of the presently disclosed devices itself, or with an instrument (e.g., guidewire) passed through a lumen of the any of the presently disclosed devices, e.g., over the wire. Further, any of the previously disclosed devices may be passed into the pericardial space through the opening in the wall of the atrial appendage, or an instrument passed through the lumen of any of the presently disclosed devices may be presented into the pericardial cavity 54. These details will depend on the procedure being performed and on the type of the previously disclosed device being employed.

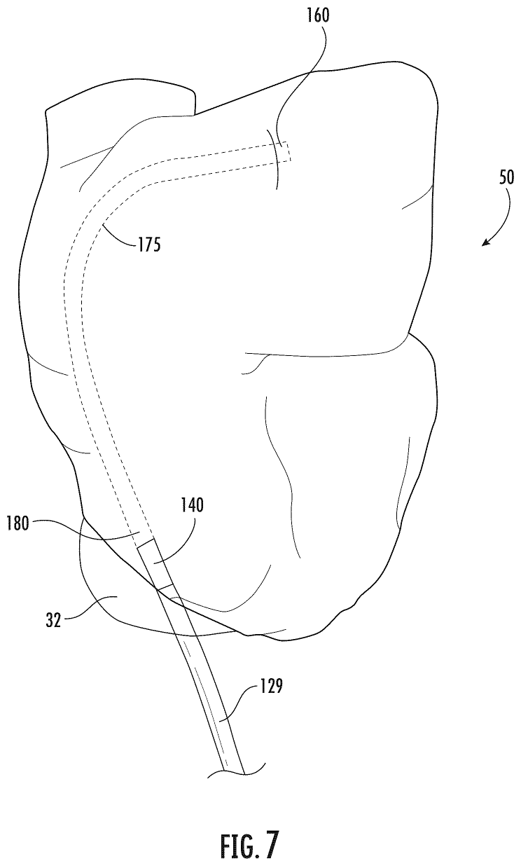

As shown in FIG. 7, any of the presently disclosed devices can be used to create a cut path of a length in a pericardium, e.g., in a parietal layer 55. Thus, a catheter 129, e.g., a steerable catheter can be employed, extending through the IVC, through the RA, and into the RAA, for example, and then into the pericardial cavity 54. The catheter 129 may have one or more steerable segments guiding any of the presently disclosed devices, with a radius of curvature of between about 1 inch and about 5 inches, with an arc length of between about 90° and about 180°. As exemplary shown in FIG. 7, any of the presently disclosed devices can be positioned in the pericardial cavity 54 and can begin a cut path 175 at a start point 160 and ends at endpoint 180 of a length. At least a portion of the parietal layer 55 of the pericardium, and the fibrous pericardium 56, and pericardial adipose tissue 59 are separated along cut path 175. The one or more incisions along the lengths, in a heart with a dysfunction treatable with the present method, cause the pericardium to separate radially about the cut line of the incision path 175, without the removal of pericardial tissue and with a reduction in pericardial restraint. One or more cut paths 175 can be made, and different cut paths, of various lengths can be used to reduce pericardial restraint. In one example, the cut path 175 and its length is pre-operatively determined. Other cut paths and lengths can be used.

In one example, the presently disclosed device further comprises at least one nerve detection device. In one example, the at least one nerve detection device is located on the flexible catheter 129. In one example, the at least one nerve detection device is located adjacent the incision device. In one example, the at least one nerve detection device is located on the dilator/introducer 115. In one example, the at least one nerve detection device is located on the cutting surface.

Any one of the presently disclosed devices can further comprises at least one nerve stimulation device. In one example, the at least one nerve stimulation device is located on the flexible catheter 129. In one example, the at least one nerve stimulation device is located adjacent the incision device. In one example, the at least one nerve stimulation device is located on dilator/introducer 115. In one example, the at least one nerve stimulation device is located on the cutting surface.

In one example, the presently disclosed devices discussed above further comprise an optical channel in the multi-lumen catheter to accommodate a lens coupled to a fiber optic cable, optionally with a light source, e.g., an LED. In one example, the presently disclosed method further comprises obtaining visual information during accessing, traversal of the pericardial cavity, exiting and/or cutting, for example, using an optical channel in the multi-lumen catheter to accommodate a lens coupled to a fiber optic cable, optionally with a light source, e.g., an LED.

A kit, comprising any one of the presently disclosed medical devices, a sheath, a guidewire, and a puncturing tip is provided.

While certain embodiments of the present disclosure have been illustrated with reference to specific combinations of elements, various other combinations may also be provided without departing from the teachings of the present disclosure. Thus, the present disclosure should not be construed as being limited to the particular exemplary embodiments described herein and illustrated in the Figures, but may also encompass combinations of elements of the various illustrated embodiments and aspects thereof.

We claim:

1. A medical device, the device comprising:
a catheter;
an incision device operably coupled to a distal end of the catheter, wherein the incision device comprises an incision channel defined along the incision device;
a first row of suction devices and a second row of suction devices, wherein the rows are positioned on opposite sides of the incision channel; and
a cutting surface disposed within the incision channel, wherein the cutting surface is structured to move between a retracted position and a deployed position,
wherein in an instance in which the cutting surface is in the retracted position, the cutting surface is disposed within the incision channel, and
wherein in an instance in which the cutting surface is in the deployed position, the cutting surface at least partially protrudes from the incision channel.

2. The medical device of claim 1, further comprising a cutting apparatus actuation mechanism structured to move the cutting surface between the retracted position and the deployed position.

3. The medical device of claim 1, wherein the first and second rows of suction devices are parallel.

4. The medical device of claim 1, wherein the first row of suction devices defines a first row length and the second row of suction devices defines a second row length, wherein at least one of the first row length or the second row length is a same length as a cutting surface length of the cutting surface.

5. The medical device of claim 1, wherein the first row of suction devices defines a first row length and the second row of suction devices defines a second row length, wherein at least one of the first row length or the second row length is a greater length than a cutting surface length of the cutting surface.

6. The medical device of claim 1, wherein the first and second row of suction devices are structured to connect to a vacuum source.

7. The medical device of claim 1, wherein the cutting surface is at least one of a blade or an electrode.

8. The medical device of claim 7, wherein the electrode is structured to receive current and/or radio frequency energy to ablate, burn, vaporize, and/or separate tissue.

9. The medical device of claim 1, wherein the catheter is steerable.

10. The medical device of claim 1, wherein at least a portion of the medical device is radiopaque.

11. The medical device of claim 1, further comprising one or more stabilizing members structured to maintain the incision device at a given location.

12. The medical device of claim 1, wherein the medical device is sterilized.

13. The medical device of claim 1, further comprising a controller engaged to the incision device, wherein the controller is structured to provide at least one of suction to first and second rows of suction devices, movement of the blade between the retracted position and the deployed position, movement of the cutting apparatus along the incision device, energy to the incision device, or movement of one or more stabilizing members.

14. A medical device, the device comprising:
a catheter;
an incision device operably coupled to a distal end of the catheter, wherein the incision device comprises an incision channel defined along the incision device;
a first row of suction devices and a second row of suction devices, wherein the rows are positioned on opposite sides of the incision channel;
one or more stabilizing members structured to maintain the incision device at a given location; and
a cutting surface disposed within the incision channel, wherein the cutting surface is structured to move between a retracted position and a deployed position,
wherein in an instance in which the cutting surface is in the retracted position, the cutting surface is disposed within the incision channel, and
wherein in an instance in which the cutting surface is in the deployed position, the cutting surface at least partially protrudes from the incision channel.

15. The medical device of claim 14, further comprising a cutting apparatus actuation mechanism structured to move the cutting surface between the retracted position and the deployed position.

16. The medical device of claim 14, wherein the first row of suction devices defines a first row length and the second row of suction devices defines a second row length, wherein at least one of the first row length or the second row length is a same length as a cutting surface length of the cutting surface.

17. The medical device of claim 14, wherein the first row of suction devices defines a first row length and the second row of suction devices defines a second row length, wherein at least one of the first row length or the second row length is a greater length than a cutting surface length of the cutting surface.

18. The medical device of claim 14, wherein the cutting surface is structured to be moveable between the retracted position and the deployed position upon placement of the incision device.

19. The medical device of claim 14, wherein the cutting surface is at least one of a blade or an electrode.

20. The medical device of claim 19, wherein the electrode is structured to receive current and/or radio frequency energy to ablate, burn, vaporize, and/or separate tissue.
